(19) United States
(12) Patent Application Publication
(10) Pub. No.: US 2025/0041234 A1
(43) Pub. Date: Feb. 6, 2025

(54) USE OF THERMOSTABLE EXOSHELLS FOR LYOPHILIZATION OF LABILE SUBSTRATES

(71) Applicant: Chester Lee Drum

(72) Inventor: Chester Lee Drum

(21) Appl. No.: 18/705,317

(22) PCT Filed: Oct. 25, 2022
(86) PCT No.: PCT/SG2022/050758

§ 371 Date: Apr. 26, 2024

(51) Int. Cl. A61K 9/50; A61K 9/19; A61K 38/43

(52) U.S. Cl. CPC A61K 9/5052; A61K 9/19; A61K 9/5089; A61K 38/43

(57) ABSTRACT

The present invention relates to a method of preparing improved powder forms of bioactive molecules. More particularly, the present invention provides a method of using engineered thermostable ferritin assemblies with excipient qualities to minimise structural and/or functional loss of labile substrates in lyophilization and storage. The present invention also provides freeze-dried compositions obtained by this method and their use in therapeutics.

### FIELD OF THE INVENTION

The present invention relates to a method of preparing improved powder forms of bioactive molecules. More particularly, the present invention provides a method of using engineered thermostable ferritin assemblies having excipient qualities to minimise structural and/or functional loss of labile substrates in lyophilization and storage. The present invention also provides freeze-dried compositions obtained by this method suitable for use in therapeutics.

### BACKGROUND OF THE INVENTION

Proteins are widely used in clinical care with applications stretching from diagnosis to therapeutics. At high concentrations, however, they exhibit enhanced viscosity, opalescence, aggregation, and immunogenicity. These properties challenge the long-term storage of protein therapeutics and significantly affect their marketability. Though aqueous formulations are considered for many of these expensive biopharmaceuticals, they are often subjected to physico-chemical degradation resulting in instability with limited shelf-life and reduced bioactivity. Additionally, they require specific conditions for their storage and transportation, making their distribution expensive and problematic. Therefore, powder formulations that are structurally and functionally stable at ambient storage conditions are needed.

Lyophilization, also known as freeze-drying, is traditionally the most convenient technique used for preparing a powder form of proteins. The objective of lyophilization is to produce a stable formulation in dry form for long-term storage and transportation. However, current lyophilization procedures have serious limitations that affect protein bioactivities. For example, freezing and dehydration stresses such as ice crystal formation, increase in solute concentration, changes in ionic strength, and extremes of pH often inactivate protein function either through unfolding or altering their structure.

Common excipients like sugars or sugar alcohols can offer protection during the freeze-drying procedure (Faghihi, H., et al., Pharm. Dev. Technol. 22, 724-732 (2017); Kadoya, S., et al., Int. J. Pharm. 389, 107-113 (2010); Chang, L. L., et al., J. Pharm. Sci. 94, 1427-1444 (2005); Mensink, M. A., et al., Eur. J. Pharm. Biopharm. 114, 288-295 (2017)), however, non-specific interactions between sugar-based stabilizers and the functional residues often impact the bioactivity of the therapeutic proteins (Kamerzell, T. J., et al., Adv. Drug Deliv. Rev. 63, 1118-1159 (2011); Hudson, K. L., et al., J. Am. Chem. Soc. 137, 15152-15160 (2015)), in addition to issues of hydrolysis and adduct formation which can affect protein stability (Li, S., et al., J. Pharm. Sci. 85, 873-877 (1996); Xu, H., et al., J. Pharm. Biomed. Anal. 32, 451-459 (2003)). Because wide ranges of protein types are not suitably addressed by current excipient technologies, novel methodologies that can stabilize complex proteins and support their prolonged storage in powder form are needed.

Current nanoparticle systems such as liposomes, metal, or polymer-based nanoparticles have improved the delivery of small molecule imaging probes and drugs to a variety of target sites. However, their ability to encapsulate bioactive proteins has met with relatively little progress, owing to the incompatibility of many proteins with the organic solvents used for the synthesis of colloidal particles. Overall, these nanoparticle systems are typically not conducive to freeze-drying stresses as, in the absence of excipients, they may undergo loss of structural integrity and the therapeutic load (Franzé, S., et al., Pharmaceutics 2018, 10, 139 (2018); Picco, A. S., et al., Nanoscale 2021, 13, 753-762 (2021)).

Accordingly, there is a need to provide methods of preparing powder formulations of labile substrates that overcome, or at least ameliorate, one or more of the disadvantages described above.

### SUMMARY OF THE INVENTION

Disclosed herein is a method of minimising structural and/or functional loss of labile substrates during storage, the method comprises encapsulating a labile substrate within an engineered nanoscale, thermostable exoshell (tES) prior to lyophilization and storage. Moreover, proteins can be stabilized without the need for additional excipients.

According to a first aspect, there is provided a method of reducing structural and/or functional loss of a labile molecule during storage, the method comprising:
(a) encapsulating a labile molecule within an engineered thermostable ferritin assembly which comprises at least one modified subunit, wherein the at least one modified ferritin subunit comprises the amino acid sequence set forth in SEQ ID NO: 1, except that the modified ferritin subunit lacks residues 165-173 of SEQ ID NO: 1, comprises a F116H substitution for pH-dependent shell disassembly/assembly, and zero or more amino acid substitutions at one or more positions selected from the group comprising E65, E128, E131, and D138 of SEQ ID NO: 1; and
(b) freeze-drying the assembly.

Advantageously, the method of the present disclosure prevents intracellular aggregation and protects the substrate from denaturation during freezing, sublimation, and storage. Significantly, the ferritin assembly by itself can operate as an effective nanoparticle lyophilization excipient and stabilize proteins in dry form without additional excipients such as sugars or sugar alcohols, examples being trehalose or sorbitol. In some embodiments, the labile molecule is a macromolecule. In some embodiments, the labile macromolecule is a polypeptide or polynucleic acid. In some embodiments, the labile macromolecule is horseradish peroxidase (HRP).

According to the invention, the ferritin subunits assemble to form a nanostructure and encapsulate a labile molecule at basic pH, whereas the nanostructure disassembles and can release an encapsulated labile molecule as the pH becomes acidic at about pH 6.

In some embodiments, the pH-dependent shell disassembly/assembly is effected in the range between pH 5.6 and pH 8.

In some embodiments, the thermostable ferritin assembly has internal charge complementarity with the labile molecule. Advantageously, the charge complementation provides stabilizing interactions for the encapsulated substrate, further enhancing storage stability.

In some embodiments, the thermostable ferritin assembly is characterized by: (i) a net internal positive (+) charge due to an amino acid substitution at any two or more positions of said ferritin assembly subunit selected from E65K, E128K, E131K, and D138A; or
(ii) a net internal neutral (+/−) charge due to amino acid substitutions E65Q and D138A of said ferritin assembly subunit; or
(iii) a net internal negative (−) charge due to zero amino acid substitutions at said ferritin assembly subunit positions E65, E128, E131 and D138.

In some embodiments, the thermostable ferritin assembly modified subunit comprises the amino acid sequence set forth in SEQ ID NO: 5, SEQ ID NO: 6, or SEQ ID NO: 7, or a variant thereof which retains the ability to assemble.

In some embodiments, prior to step (b) the assembly is chromatographed on a column equilibrated with a volatile salt buffer at about pH 8.0.

In some embodiments, the said volatile salt buffer is ammonium bicarbonate or formate buffer. In a further embodiment, the buffer is preferably about 10 mM ammonium bicarbonate.

In some embodiments, the freeze-drying is performed for a period of about 48 h or more, depending on the volume of the assembly.

According to a second aspect, there is provided a kit comprising thermostable ferritin assembly modified subunits, as defined herein, to be used in any of the methods of the first aspect.

According to a third aspect, there is provided a freeze-dried composition comprising a labile molecule encapsulated within an engineered thermostable ferritin assembly as defined in any aspect of the invention.

In some embodiments, there is provided a freeze-dried composition produced by any of the methods of the present disclosure.

In some embodiments, the freeze-dried composition of the present disclosure is for use in the prophylaxis or treatment of disease in a subject.

In some embodiments of the freeze-dried composition, the encapsulated labile molecule comprises an enzyme for conversion of a prodrug. In some embodiments, the freeze-dried composition of the present disclosure further comprises one or more additional therapeutic agents.

In a fourth aspect, there is provided the use of the freeze-dried composition of the present disclosure in the manufacture of a medicament for the prophylaxis or treatment of a disease.

According to a fifth aspect, there is provided a method of treatment or prophylaxis comprising administering to a subject in need of such treatment or prophylaxis an efficacious amount of any one of the freeze-dried compositions as described in any aspect of the invention.

As described herein, the methods of the present disclosure advantageously stabilize labile macromolecules during freeze-drying and storage.

### BRIEF DESCRIPTION OF THE FIGURES

The accompanying drawings illustrate a disclosed embodiment and serve to explain the principles of the disclosed embodiment. It is to be understood, however, that the drawings are designed for purposes of illustration only, and not as a definition of the limits of the invention.

FIG. 1 shows a schematic of an embodiment of the present invention, depicting the nanoencapsulation of a substrate of interest, horseradish peroxidase (HRP), within a thermostable exoshell (tES), followed by lyophilization and storage.

FIGS. 2A-2D show the results of thermostable exoshells (tES) expression. FIG. 2A Comparison of wet weight of bacterial cell mass produced in LB, TB, and 2XYT media.

FIG. 2B Comparison of overall yields of tES expressed in LB, TB, and 2XYT media. FIG. 2C Purification of tES by size-exclusion chromatography (SEC). (Note: Fractions of the tES peak (rectangle box) were analyzed for purity in SDS-PAGE, pooled, and concentrated for various experiments). FIG. 2D SDS-PAGE analysis of purified tES compared to lysate (L).

FIGS. 3A-3C show the results of re-purification of tES. FIG. 3A RP-HPLC chromatography of SEC purified tES on a linear gradient of 0-100% solvent B. The elution was monitored at 214 nm and 280 nm. FIG. 3B The ESI-MS of tES showing multiple peaks of mass/charge (m/z) ratio ranging from +13 to +29 charges. FIG. 3C The mass of tES was determined to be 19286.52 Da.

FIG. 4 depicts a bar graph showing the effects of tES on cell viability.

FIGS. 5A-5C show the cake appearance of the following freeze-dried proteins. FIG. 5A tES; FIG. 5B tES-HRP; FIG. 5C HRP.

FIGS. 6A-6D show the results of the characterization of freeze-dried tES. FIG. 6A Size-exclusion chromatogram of freeze-dried tES at pH 8.0 after storage at days 1 and 30 at room temperature. The pre-lyophilized samples were considered as control. FIG. 6B Comparison of concentrations of freeze-dried tES after storage at days 1 and 30. The pre-lyophilized samples were considered as control. FIG. 6C Size-exclusion chromatogram of the freeze-dried tES subunits after storage at day 30. The subunits were either reconstituted in 50 mM Tris-HCl pH 8.0 for their shell assembly or 50 mM sodium acetate buffer pH 5.8. FIG. 6D CD spectra of freeze-dried tES after storage at days 1 and 30 at room temperature. Compared to pre-lyophilized samples, tES exhibited double minima at 210 nm and 225 nm typical of α-helical structure (n=3, mean±SD).

FIGS. 7A-7H show the results of nanoencapsulation of HRP. FIG. 7A Size-exclusion chromatogram of free HRP at pH 8.0. FIG. 7B Size-exclusion chromatogram of tES at pH 8.0. FIG. 7C Size-exclusion chromatogram of tES-HRP at pH 8.0. Each fraction was analyzed for HRP activity and the data overlaid on the respective chromatogram (Note: Fractions of the tES-HRP peak (rectangle box) were pooled and concentrated for various experiments). FIG. 7D Hydrodynamic diameter measurement of free HRP from DLS experiments. FIG. 7E Hydrodynamic diameter measurement of tES from DLS experiments. FIG. 7F Hydrodynamic diameter measurements of tES-HRP from DLS experiments. FIG. 7G SDS-PAGE analysis of nanoencapsulation. Lane 1 (M-protein marker), Lane 2 (tES) showing single band corresponding to ˜20 kDa of subunit, Lane 3 (HRP) showing a single band of ˜44 kDa, and Lane 4 (tES-HRP) showing two bands corresponding to subunit (˜20 kDa) and HRP (˜44 kDa). The quantification analysis of lane 4 through densitometry exhibited a 12-time higher intensity for tES (band intensity of 4.28×10⁸) compared to HRP (band intensity of 3.55×10⁷). FIG. 7H Thermogravimetric analysis (TGA) of the lyophilized proteins to determine the residual moisture content. The TGA curves of HRP, tES, and tES-HRP are shown.

FIGS. 8A-8C depict the results of the functional and structural characterization of freeze-dried tES-HRP. FIG. 8A Enzyme activities of freeze-dried free or encapsulated HRP at different storage conditions: room temperature (RT), 4° C., and −20° C. for day 1, week 1, and month 1. The enzyme activities were compared to pre-lyophilized samples (control). FIG. 8B CD spectra of freeze-dried HRP at different storage conditions in comparison to the pre-lyophilized sample (control). FIG. 8C CD spectra of freeze-dried tES at different storage conditions in comparison to the pre-lyophilized sample (control). (n=3, mean±SD). Statistical significance (** p≤0.01, *** p≤0.001, **** p≤0.0001) was determined by one-way ANOVA.

FIG. 9 shows a graph of the time-dependent activity of freeze-dried tES-HRP assayed at 652 nm.

FIGS. 10A-10I depict the stability of lyophilized tES after storage for 8 months in three temperature conditions: Room Temperature (RT), 4° C., −20° C., with or without excipients (60 mM trehalose or 60 mM sorbitol). FIG. 10A Size-exclusion chromatogram of the freeze-dried tES at pH 8.0, after storage without any added excipients. FIG. 10B Size-exclusion chromatogram of the freeze-dried tES at pH 8.0, after storage with 60 mM trehalose. FIG. 10C Size-exclusion chromatogram of the freeze-dried tES at pH 8.0, after storage with 60 mM sorbitol. FIG. 10D Size-exclusion chromatogram of the freeze-dried tES at pH 5.6, after storage without any added excipients. FIG. 10E Size-exclusion chromatogram of the freeze-dried tES at pH 5.6, after storage with 60 mM trehalose. FIG. 10F Size-exclusion chromatogram of the freeze-dried tES at pH 5.6, after storage with 60 mM sorbitol. FIG. 10G CD spectra of freeze-dried tES with or without added excipients after storage at room temperature (RT). FIG. 10H CD spectra of freeze-dried tES with or without added excipients after storage at 4° C. FIG. 10I CD spectra of freeze-dried tES with or without added excipients after storage at −20° C.

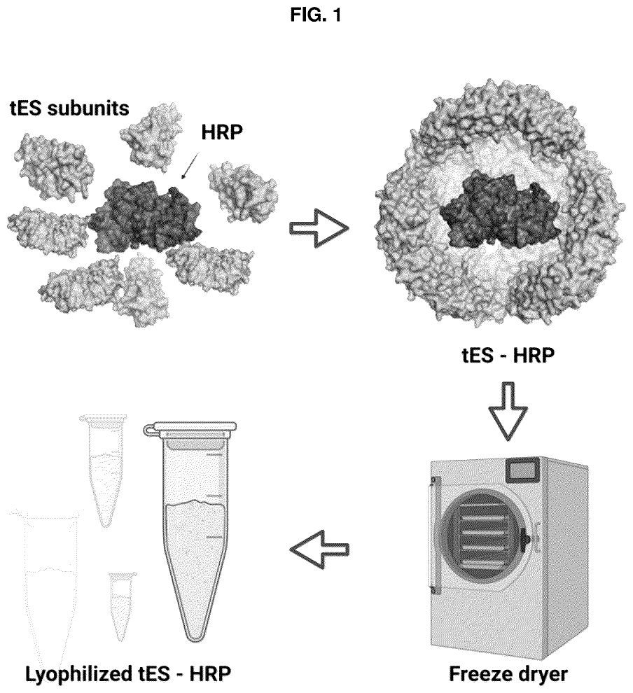

### DETAILED DESCRIPTION OF THE INVENTION

Disclosed herein is a method for optimising storage of labile substrates, the method comprises encapsulating labile substrates within the engineered thermostable ferritin assemblies (tES) of the present disclosure, followed by lyophilization and storage. Preferably, each protective tES provides internally facing, stabilizing interactions such as charge complementarity for the encapsulated substrate to further enhance its storage stability. Without being bound by theory, it appears that tight steric and electrostatic complementarity to an encapsulated substrate advantageously provides further stabilizing forces that increases the resistance of the substrate against external degradation-causing conditions faced during lyophilization and storage.

A description of exemplary, non-limiting, embodiments of the invention follows.

The present invention is based, in part, on the development of a method for optimising storage of labile substrates, comprising encapsulating labile substrates such as protein strands, nucleic acids or other macromolecules into engineered, aqueous cavities using nanoscale, thermostable exoshells (tES) and freeze-drying the complex prior to storage. Preferably, each protective tES provides internally facing, stabilizing interactions for the encapsulated substrate to enhance storage stability.

As used herein, “a” or “an” may mean one or more than one unless indicated to the contrary or otherwise evident from the context.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

The inventors have modified a thermostable ferritin protein nanocage from the hyperthermophilic bacterium Archaeoglobus fulgidus (AfFtn) to accommodate host proteins and provide charge-charge stabilization (Deshpande, S., et al., Nat. Commun. 8, 1-8 (2017)). The engineered tES has an 8 nm aqueous cavity that can theoretically accommodate molecules up to a volume of ˜306 nm³ (Guex, N., et al., Electrophoresis 30, S162-S173 (2009); Voss, N. R., and Gerstein, M., Nucleic Acids Res. 38, W555-W562 (2010)). The shell, an assembly of 24 subunits, contains four 4.5 nm surface pores, which allow for easy permeation of solutes. Further, the encapsulated proteins are protected from proteases or extreme denaturing conditions.

The tES described herein are highly stable even in low salt concentrations and engineered with net positive, neutral, or negative charged internal environments. The tES described herein are also engineered to disassemble and assemble in a pH-dependent manner, which facilitates the controlled release of encapsulated proteins. As demonstrated herein, the tES used in the method of the present disclosure serve as effective nanoparticle lyophilization excipients and advantageously stabilize macromolecules in dry form.

### Ferritin Assembly Composition

Disclosed herein is an engineered thermostable ferritin assembly comprising at least one modified ferritin subunit, wherein the at least one modified ferritin subunit comprises the amino acid sequence set forth in SEQ ID NO: 1, except that the modified ferritin subunit lacks residues 165-173 of SEQ ID NO: 1, comprises a F116H substitution for pH-dependent shell disassembly/assembly, and zero or more amino acid substitutions at one or more positions selected from the group comprising E65, E128, E131, and D138 of SEQ ID NO: 1.

As used herein, “ferritin assembly” is used synonymously with ferritin, a known structure in the art, that comprises 24 subunits (ferritin subunits), each subunit having a defined size (e.g., ˜20 kDa for AfFtn) and structural motif (e.g., a four-helix bundle structural motif for AfFtn). The native ferritin assembly has a defined external diameter (e.g., 12 nm for AfFtn) and a defined internal cage (e.g., 8 nm for AfFtn). Ferritin has been well-characterized in the art (e.g., Johnson, E., et al., Structure 13, 637-648 (2005)).

As used herein, a ferritin subunit refers, as noted above, to one of 24 subunits that form the ferritin assembly. Subunits of the ferritin assembly have a defined structure, comprising a four-helix bundle motif. In AfFtn, each subunit is ˜20 kDa and contains a four-helix bundle structural motif. An AfFtn wildtype subunit has the amino acid sequence set forth in SEQ ID NO: 1 (Table 1).

TABLE 1
Amino acid sequence of AfFtn subunits

| | |
|---|---|
| SEQ ID NO: 1 | MASISEKMVE ALNRQINAEI YSAYLYLSMA |
| AfFtn wildtype subunit | SYFDSIGLKG FSNWMRVQWQ EELMHAMKMF |
| (unstructured C-terminal | DFVSERGGRV KLYAVEEPPS EWDSPLAAFE |
| sequence underlined) | HVYEHEVNVT KRIHELVEMA MQEKDFATYN |
| | FLQWYVAEQV EEEASALDIV EKLRLIGEDK |
| | RALLFLDKEL SLRQFTPPAE EEK |
| SEQ ID NO: 2 | MASISEKMVE ALNRQINAEI YSAYLYLSMA |
| AfFtn subunit with truncation | SYFDSIGLKG FSNWMRVQWQ EELMHAMKMF |
| of unstructured C-terminus | DFVSERGGRV KLYAVEEPPS EWDSPLAAFE |
| | HVYEHEVNVT KRIHELVEMA MQEKDFATYN |
| | FLQWYVAEQV EEEASALDIV EKLRLIGEDK |
| | RALLFLDKEL SLRQTS |
| SEQ ID NO: 3 | MASISEKMVE ALNRQINAEI YSAYLYLSMA |
| AfFtn net positive variant | SYFDSIGLKG FSNWMRVQWQ EELMHAMKMF |
| subunit with truncation of | DFVSKRGGRV KLYAVEEPPS EWDSPLAAFE |
| unstructured C-terminus | HVYEHEVNVT KRIHELVEMA MQEKDFATYN |
| | FLQWYVAKQV KEEASALAIV EKLRLIGEDK |
| | RALLFLDKEL SLRQTS |
| SEQ ID NO: 4 | MASISEKMVE ALNRQINAEI YSAYLYLSMA |
| AfFtn net neutral variant | SYFDSIGLKG FSNWMRVQWQ EELMHAMKMF |
| subunit with truncation of | DFVSQRGGRV KLYAVEEPPS EWDSPLAAFE |
| unstructured C-terminus | HVYEHEVNVT KRIHELVEMA MQEKDFATYN |
| | FLQWYVAEQV EEEASALAIV EKLRLIGEDK |
| | RALLFLDKEL SLRQTS |
| SEQ ID NO: 5 | MASISEKMVE ALNRQINAEI YSAYLYLSMA |
| AfFtn subunit with truncation | SYFDSIGLKG FSNWMRVQWQ EELMHAMKMF |
| of unstructured C-terminus | DFVSERGGRV KLYAVEEPPS EWDSPLAAFE |
| and F116H mutation | HVYEHEVNVT KRIHELVEMA MQEKDHATYN |
| | FLQWYVAEQV EEEASALDIV EKLRLIGEDK |
| | RALLELDKEL SLRQTS |
| SEQ ID NO: 6 | MASISEKMVE ALNRQINAEI YSAYLYLSMA |
| AfFtn net neutral variant | SYFDSIGLKG FSNWMRVQWQ EELMHAMKMF |
| subunit with truncation of | DFVSQRGGRV KLYAVEEPPS EWDSPLAAFE |
| unstructured C-terminus | HVYEHEVNVT KRIHELVEMA MQEKDHATYN |
| and F116H mutation | FLQWYVAEQV EEEASALAIV EKLRLIGEDK |
| | RALLFLDKEL SLRQTS |
| SEQ ID NO: 7 | MASISEKMVE ALNRQINAEI YSAYLYLSMA |
| AfFtn net positive variant | SYFDSIGLKG FSNWMRVQWQ EELMHAMKMF |
| subunit with truncation of | DFVSKRGGRV KLYAVEEPPS EWDSPLAAFE |
| unstructured C-terminus | HVYEHEVNVT KRIHELVEMA MQEKDHATYN |
| and F116H mutation | FLQWYVAKQV KEEASALAIV EKLRLIGEDK |
| | RALLFLDKEL SLRQTS |

As used herein, a “modified ferritin subunit” refers to a ferritin subunit that lacks an unstructured carboxy-terminal sequence that a wildtype ferritin subunit possesses. That is, the modified ferritin subunit comprises a deletion of amino acids 165-173 of SEQ ID NO: 1. For use according to the invention, the subunit sequence is truncated at position 164 of SEQ ID NO: 1 and comprises a F116H substitution, as set forth in SEQ ID NO: 5 (Table 1).

As used herein, an “engineered ferritin assembly” refers to a ferritin assembly formed with at least one modified ferritin subunit, as described herein with reference to Deshpande, S., et al., Nat. Commun. 8, 1-8 (2017). The engineered ferritin assembly is sometimes referred to as a “nanoshell” or “nanocage” or “nanoencapsulator”, “NE” or “thermostable exoshell” or “tES”. An example of a modified ferritin subunit comprising residues 1-164 is set forth in SEQ ID NO: 2. As those of skill in the art would recognize, residues can be present beyond the last residue in the modified ferritin subunit that results from, e.g., cloning sites, that do not impact the engineered ferritin assembly function. For example, as shown in Table 1, AfFtn subunit with a truncation of the unstructured C-terminus comprise the residues “TS” that resulted from a cloning site (restriction site) in the cloning plasmid. Table 1 summarizes the sequences described herein. Removal of the unstructured carboxy-terminal sequence not only frees up space inside the engineered ferritin assembly but also imparts stability to the assembly in low salt concentrations (e.g. below 30 mM NaCl).

As described herein, the engineered ferritin assembly can be designed for controlled release of the encapsulated polypeptide. In certain embodiments, the modified ferritin subunit comprises a substitution at position F116 of SEQ ID NO: 1 of AfFtn. In certain embodiments, the at least one modified ferritin subunit comprises the substitution F116H; an example of which is set forth in SEQ ID NO: 5. An engineered ferritin assembly with F116H mutation is shown to reversibly dissociate at acidic pH (i.e. below pH 7.0), preferably from about pH 4.0, more preferably at about pH 5.8, and stably reassemble at basic pH (i.e. above pH 7.0), preferably at about pH 8.0.

The modified ferritin subunit can comprise one or more further modifications (e.g., amino acid substitutions) to its amino acid sequence. As described herein, at least one ferritin subunit can be modified to contribute to a net charge (e.g., positive, negative, or neutral) of the interior surface of the ferritin assembly. Methods of determining which residues to modify within a ferritin subunit to achieve a ferritin assembly possessing a net positive, negative, or neutral interior charge are known in the art, as described herein.

In certain embodiments, the ferritin subunit can be modified such that the ferritin assembly possesses a net positive interior charge. In certain embodiments, the at least one modified ferritin subunit comprises an amino acid substitution at any one or more positions selected from E65, E128, E131, and D138 of SEQ ID NO: 1 of an AfFtn subunit. In certain embodiments, the at least one modified ferritin subunit comprises any one or more substitutions selected from E65K, E128K, E131K, and D138A; an example of which is set forth in SEQ ID NO: 7.

In certain embodiments, the ferritin subunit can be modified such that the ferritin assembly possesses a net neutral interior charge; an example of which is set forth in SEQ ID NO: 6.

As described herein, an engineered ferritin assembly formed from native AfFtn ferritin subunit truncations possesses a net negative charge; an example of which is set forth in SEQ ID NO: 5.

As used herein, “protein” and “polypeptide” are used interchangeably to denote a polymer of at least two amino acids covalently linked by an amide bond, regardless of length or post-translational modification (e.g., glycosylation or phosphorylation). The term “protein” encompasses a naturally-occurring as well as artificial (e.g., engineered or variant) full-length protein as well as a functional fragment of the protein.

The term “functional fragment” refers to a portion of a protein that retains some or all of the activity or function (e.g., biological activity or function, such as enzymatic activity) of the full-length protein, such as, e.g., the ability to bind and/or interact with or modulate another protein or nucleic acid. The functional fragment can be any size, provided that the fragment retains, e.g., the ability to bind and interact with another protein or nucleic acid.

Preferably, the thermostable ferritin assembly modified subunit comprises the amino acid sequence set forth in SEQ ID NO: 5, SEQ ID NO: 6, or SEQ ID NO: 7, or a variant thereof which retains the ability to assemble. The choice of subunit sequence may depend on the desired charge complementarity required between the interior of the assembly and its encapsulated labile molecule.

In some embodiments, the modified ferritin assembly comprising the substrate undergo buffer exchange after encapsulation, to prepare for lyophilization. Buffer exchange is typically an essential step in preparing biological materials for downstream applications/analyses and enabling storage. The methods of buffer exchange are well known in the art. For example, buffer exchange can be performed using dialysis, diafiltration and column chromatography. In some embodiments, the modified ferritin assembly is chromatographed on a column equilibrated with a volatile salt buffer. Preferably, the chromatography is carried out at about pH 8.0. Preferably, the volatile buffer is ammonium bicarbonate or formate buffer. Preferably, the buffer is about 10 mM ammonium bicarbonate. Preferably, the assembly is chromatographed on a column equilibrated with a volatile salt buffer at about pH 8.0, prior to freeze-drying.

As used herein, the terms “lyophilization” and “freeze-drying” are used interchangeably and refer to a process in which water is removed from a substance after it is frozen and placed under a vacuum, allowing the ice to change directly from solid to vapour without passing through a liquid phase. The process generally comprises three separate, unique and interdependent processes; freezing, primary drying (sublimation) and secondary drying (desorption). Lyophilization is a commonly used technique in the biomedical and pharmaceutical industry for drugs, vaccines, antibodies, and other biological materials, and it would be appreciated that the general lyophilization technology is well known in the art.

The time required to lyophilize a substrate depends on many factors such as, but not limited to, the type of substrate, its size and volume, the surface area, and the system employed etc. Exemplary time periods for use in the method of the present disclosure is about less than 24 h, about 24 h, from about 24 h to about 48 h, about 48 h, or more than 48 h. In some embodiments, the encapsulated substrate is lyophilized for about 48 h. Preferably, the encapsulated substrate is lyophilized for about 48 h or more, depending on the volume of the assembly.

Advantageously, the methods described herein enhance the stability of labile molecules in storage without adding excipients. Without being bound by theory, it appears that the engineered ferritin assembly as used in the methods described herein provides an optimal protective barrier against external environmental stressors encountered during lyophilization and storage. Encapsulating a polypeptide substrate with the engineered ferritin assembly as described in the method of the present disclosure increases the polypeptide's resistance to structural and functional degradation that would otherwise occur in the polypeptide if lyophilized and stored without encapsulation by the engineered ferritin assembly.

As would be well appreciated by a person skilled in the art, the biological function of a polypeptide/protein is closely linked to its folding and structure. Therefore, as used herein, the term “reducing structural loss” refers to minimising any changes in the structure/conformation of a polypeptide, minimising degradation of its intended structure, increasing its resistance to factors that may affect its intended structure or a combination thereof, which results in its improved ability to maintain the intended structure over a time period. Similarly, as used herein, the term “reducing functional loss” refers to minimising any changes in the intended function of a polypeptide, minimising the effectiveness of its intended function, increasing its resistance to factors that may affect its intended function or a combination thereof, which results in its improved ability to maintain the intended function over a time period.

As used herein, the term “substrate” refers to any molecule that may be encapsulated by the tES of the present disclosure. Preferably, the substrate is a labile substrate. Preferably, the labile substrate is a labile molecule. As used herein, a “labile molecule” refers to any molecule that is sensitive to any change(s) in its external environment such as but not limited to temperature, concentration, ionic charge and pH, wherein said change(s) in its external environment may affect its intended structure, conformation and/or function.

In some embodiments, the labile molecule may be a polypeptide, a nucleic acid or a macromolecule. In some embodiments, the labile molecule may have a therapeutic application. In some embodiments, the labile molecule is a macromolecule. In some embodiments, the labile molecule may be a macromolecule comprising an enzyme for conversion of a prodrug. In some embodiments, the labile molecule is horseradish peroxidase (HRP).

As used herein, the term “polynucleic acid” refers to a polymer comprising multiple nucleotide monomers (e.g., ribonucleotide monomers or deoxyribonucleotide monomers). “Polynucleic acid” includes, for example, genomic DNA, cDNA, RNA, and DNA-RNA hybrid molecules. Polynucleic acid molecules can be naturally occurring, recombinant, or synthetic. In addition, polynucleic acid molecules can be single-stranded, double-stranded or triple-stranded. In certain embodiments, polynucleic acid molecules can be modified. In the case of a double-stranded polymer, “polynucleic acid” can refer to either or both strands of the molecule.

In another aspect, there is provided a freeze-dried composition comprising a labile molecule encapsulated within an engineered thermostable ferritin assembly as described in the present disclosure.

In yet another aspect, there is provided a freeze-dried composition produced by any of the methods as described in the present disclosure. Advantageously, the freeze-dried composition of the present disclosure has improved stability and is suitable for prolonged storage. In some embodiments, the labile molecule in the freeze-dried composition is a macromolecule comprising an enzyme for conversion of a prodrug. In some embodiments, the freeze-dried composition comprises one or more additional therapeutic agents.

In another aspect, there is provided a kit, or use of a kit, in a method of reducing structural and/or functional loss of labile molecules as described herein, the kit comprises at least one thermostable ferritin assembly modified subunit as defined in the present disclosure. In some embodiments, the kit may comprise a means to encapsulate a labile molecule. In some embodiments, the kit may also comprise a means to lyophilize said encapsulated labile molecule. In some embodiments, the kit may further comprise a means to store the lyophilized encapsulated molecules. In some embodiments, the kit may also comprise a device or system to reconstitute the lyophilized molecules.

Methods of reconstituting freeze-dried compositions are well known in the art. It would be appreciated by a person of skill in the art that a freeze-dried composition may be reconstituted easily with diluents such as sterile water or saline solution.

In another aspect, there is provided a freeze-dried composition of the present disclosure for use in the prophylaxis or treatment of disease in a subject.

In another aspect, there is provided a use of the freeze-dried composition as described herein in the manufacture of a medicament for the prophylaxis or treatment of a disease.

In some embodiments, the disease relates to diseased heart cells, diseased liver cells, diseased neurons, or diseased immune cells. In certain embodiments, the disease is cancer.

In another aspect, there is also provided a method of treatment or prophylaxis comprising administering to a subject in need of such treatment or prophylaxis an efficacious amount of a freeze-dried composition of the present disclosure.

In some embodiments, the freeze-dried composition may be administered in combination with one or more additional therapeutic agents. When administered in a combination therapy, the freeze-dried composition may be administered before, after or concurrently with the other therapy. When co-administered simultaneously (e.g., concurrently), the freeze-dried composition and other therapy can be in separate formulations or the same formulation. Alternatively, the freeze-dried composition and other therapy can be administered sequentially, as separate compositions, within an appropriate time frame as determined by a skilled clinician (e.g., a time sufficient to allow an overlap of the pharmaceutical effects of the therapies). In some embodiments, the freeze-dried composition is first reconstituted prior to use and/or administration for therapy. In certain embodiments, the disease is cancer. In certain embodiments, the freeze-dried composition comprising the engineered ferritin assembly is a vaccine.

In certain embodiments, the freeze-dried composition can be reconstituted and can be delivered into a cell (e.g., in vivo delivery) in a formulation comprising the reconstituted composition and one or more pharmaceutically acceptable carriers or excipients. Suitable pharmaceutical carriers typically will contain inert ingredients that do not interact with the agent or nucleic acid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's lactate and the like. Formulations can also include small amounts of substances that enhance the effectiveness of the active ingredient (e.g., emulsifying agents, solubilizing agents, pH buffering agents, wetting agents). Methods of encapsulation compositions (such as in a coating of hard gelatin or cyclodextran) are known in the art. For inhalation, the agent can be solubilized and loaded into a suitable dispenser for administration (e.g., an atomizer or nebulizer or pressurized aerosol dispenser).

For in vivo delivery, the reconstituted composition comprising engineered ferritin assembly that encapsulates a polypeptide can be delivered to a subject in need thereof by a variety of routes of administration including, for example, oral, dietary, topical, transdermal, or parenteral (e.g., intra-arterial, intravenous, intramuscular, subcutaneous injection, intradermal injection) routes of administration. Administration can be local or systemic. The actual dose of a therapeutic polypeptide encapsulated by an engineered ferritin assembly and treatment regimen can be determined by a skilled physician, taking into account the nature of the condition being treated, and patient characteristics.

Unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. “About” in reference to a numerical value generally refers to a range of values that fall within ±10%, in some embodiments ±5%, in some embodiments ±1%, in some embodiments ±0.5% of the value unless otherwise stated or otherwise evident from the context. In any embodiment in which a numerical value is prefaced by “about”, an embodiment in which the exact value is recited is provided. Where an embodiment in which a numerical value is not prefaced by “about” is provided, an embodiment in which the value is prefaced by “about” is also provided. Where a range is preceded by “about”, embodiments are provided in which “about” applies to the lower limit and to the upper limit of the range or to either the lower or the upper limit, unless the context clearly dictates otherwise. Where a phrase such as “at least”, “up to”, “no more than”, or similar phrases, precedes a series of numbers, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated.

Unless otherwise indicated, all technical and scientific terms used herein have the same meaning as commonly understood to one of ordinary skill in the art to which the invention belongs.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1: Materials and Methods

### Expression of tES and Encapsulation of Labile Molecules

The cloning and expression of tES were executed as earlier described in Deshpande, S., et al., Nat. Commun. 8, 1-8 (2017), incorporated herein by reference in its entirety. Briefly, pRSF1b vectors carrying the tES gene (preferably encoding a subunit polypeptide comprising the amino acid sequence set forth in SEQ ID NO: 5, 6, or 7) were transformed into non-heat shock HIT-21 competent cells (Real Biotech Corporation, Taipei, Taiwan) and cultured on Luria-Bertani (LB) agar (Axil Scientific, Singapore) plates supplied with 25 μg/mL kanamycin (Thermo Fisher Scientific, Waltham, MA, USA). Once the positive colonies were confirmed through sequencing, the glycerol stocks were prepared and stored at −80° C. For the expression of tES, a starter culture was prepared by inoculating 100 μL glycerol stock in 100 mL of LB broth, Terrific broth (TB; Sigma, St. Louis, MO, USA), or 2XYT (Sigma, St. Louis, MO, USA) media with 50 μg/mL kanamycin. Following overnight incubation at 37° C., 25 mL of the starter culture was used to inoculate 1 L of the respective media and allowed to grow until an absorbance (OD600) of 0.5-0.6 was reached. Protein expression was then induced with 0.4 mM IPTG (Axil Scientific, Singapore). After 5 h of incubation at 37° C., cells were centrifuged at 12,000 rpm for 20 min and the wet weight was measured.

### Purification of tES and its Subunits

The harvested cell pellets from the three different media were resuspended in lysis buffer (50 mM Tris-HCl, 200 mM NaCl, 0.1% Triton-X 100 pH 8.0), sonicated, and centrifuged to obtain the lysate. The lysate was heated at 70° C. for 10 min and the precipitated sample was centrifuged at 12,000 rpm for 20 min and size-fractionated by size-exclusion chromatography (SEC) using a Superdex 200 (Cytiva, Marlborough, MA, USA) column equilibrated with 50 mM Tris-HCl pH 8.0. The purity of tES fraction was analyzed using sodium dodecyl sulfate-polyacrylamide gel electrophoresis (SDS-PAGE). Alternatively, the SEC purified tES fractions were re-chromatographed by reverse phase-high performance liquid chromatography (RP-HPLC) on a Phenomenex C₄ column equilibrated with 0.1% trifluoroacetic acid (TFA). The bound proteins were eluted using a linear gradient of 0-100% solvent B (80% acetonitrile in 0.1% TFA) and the elution was monitored at both 215 and 280 nm. The purity and mass of the eluted protein were determined by electrospray ionization mass spectrometry (ESI-MS) using an LCQ Fleet Ion Trap mass spectrometer (Thermo Fisher Scientific, Waltham, MA, USA). Mass analysis involved injection of 25 μL of the sample at 200 μL/min flow rate into the ion source. Sheath gas flow rate of 30, auxiliary gas flow rate of 5 at a spray voltage of 4.5 KV, and capillary temperature of 350° C. were used for ionizing the sample. The ion-trap detector was set in the positive mode with a mass/charge range of 600-2000. Qual browser, Thermo Xcalibur software was used for analysis and Pro-mass software (Thermo Fisher Scientific, Waltham, MA, USA) was used for reconstructing the mass spectrum. The concentration of the purified shell proteins was measured on a Nanodrop (DeNovix, Wilmington, DE, USA) as per Beer-Lambert's equation by measuring the absorbance at 280 nm and using the molar extinction coefficient of ϵ280=814080 M⁻¹·cm⁻¹. Similarly, the tES subunits were purified from the bacterial cell pellet of TB culture using the lysis buffer (50 mM sodium acetate 0.1% Triton-X 100 pH 5.8). The protein concentration was determined as mentioned above but using the molar extinction coefficient of ϵ280=33920 M⁻¹·cm⁻¹ for the subunits.

Nanoencapsulation of HRP within tES

Purified tES was mildly acidified for shell disassembly in 50 mM Tris-citrate pH 5.8. The disassembled subunits were separated in SEC using the same buffer. The collected subunits were mixed with HRP (Thermo Fisher Scientific, Waltham, MA, USA) at a 10:1 molar ratio and incubated for 30 min. The pH of the mixture was increased to 8.0 using 2 M NaOH and the assembled tES-HRP was separated from free HRP using SEC with 10 mM ammonium bicarbonate pH 8.0 buffer. The activity of the free or encapsulated HRP was quantified using the 1-step™ ultra TMB (Thermo Fisher Scientific, Waltham, MA, USA) assay for peroxidase wherein, the HRP oxidizes 3,3′,5,5′-tetramethylbenzidine (TMB) substrate in the presence of hydrogen peroxide to form a blue color compound. After quenching the reaction with 2 M sulphuric acid, the absorbance was recorded at 450 nm.

### Size Determination

Dynamic light scattering (DLS) measurements were performed at room temperature (RT) using a Zetasizer Pro (Malvern Instruments Ltd., Malvern, UK). All the protein solutions used were syringe filtered by using Minisart 0.2 μm syringe filters (Sartorius, Göttingen, Germany). Stock samples of individual proteins (tES, tES-HRP, and HRP) at final concentrations of 1 mg/mL were prepared in 50 mM Tris-HCl pH 8.0. The protein concentrations were optimized in preliminary experiments to obtain reliable measurements. The ZS Xplorer software suite (Malvern Instruments Ltd., Malvern, UK) was used to analyze the acquired correlation function and to derive the translational diffusion coefficient (D). Assuming particle sphericity, the hydrodynamic diameter (d_{H}) of the diffusing particles was calculated using the Stokes-Einstein equation: d_{H}=KT/3πηD where k is Boltzmann's constant, T is the absolute temperature, and η is the viscosity of the solvent.

### CD Spectroscopy

Far-UV CD spectra (260-190 nm) were acquired using a Jasco J-1100 CD spectrometer (Jasco, Tokyo, Japan). Protein samples (tES and HRP) at 0.2 mg/mL were prepared in 10 mM phosphate buffer and measurements were carried out at room temperature (RT) using a 1 mm stoppered quartz cuvette. Each spectrum was obtained after averaging three scans of the spectral range (Spectra Manager, Jasco, Tokyo, Japan). Spectra of buffer blanks were measured prior to the samples and were subtracted from the sample CD spectra.

### Lyophilization Experiments

Before lyophilization, all the proteins (tES, tES-HRP, and HRP) were chromatographed on an SEC column equilibrated with 10 mM ammonium bicarbonate pH 8.0 and frozen at −80° C. with an exception of tES subunits that were frozen in 10 mM ammonium formate pH 5.8 (Franks, F., Eur. J. Pharm. BioPharmaceutics 45, 221-229 (1998)). They were subjected to freeze-drying for 48 h using an Alpha 1-2 LDplus lyophilizer (Christ, Osterode am Harz, Germany) at a condenser temperature of −52° C. and a vacuum pressure of 1.0 mbar. Lyophilized protein powders of tES or its subunits were stored at RT to evaluate their stability. On days 1 and 30, the reconstituted tES samples (1 mg/mL) were analyzed on SEC, pH 8.0 to assess their ability to elute as shells, considering pre-lyophilized samples as the control. Likewise, the tES subunits after 30-day storage were reconstituted at pH 8.0 or 5.8 to analyze their ability to elute as shells or subunits on SEC, respectively. Further, the dry powder of tES-HRP (10 mg/mL) or free HRP (equivalent to the activity of 10 mg/mL tES-HRP) were incubated at RT, 4° C., and −20° C. for up to 1 month after which they were reconstituted in 10 mM ammonium bicarbonate buffer. The HRP activity was quantified using TMB assay as described previously and the percent activity was determined considering pre-lyophilization activity as 100%. Additionally, the time-dependent activity of freeze-dried tES-HRP was determined using the TMB assay measured at 652 nm. Finally, the CD spectra were acquired to study the conformational stability of the proteins post-freeze-drying and storage. All the enzyme activity experiments were conducted independently in triplicates.

### Thermogravimetric Analysis

The residual moisture content in the freeze-dried samples was determined using Thermogravimetric analysis (TGA) on a Discovery TGA (TA Instruments, New Castle, DE, USA). Samples from three independent lyophilized vials were heated from room temperature to 1000° C. at a rate of 10° C./min under a continuous nitrogen purge and the results were analyzed using the Trios v4 software (TA Instruments, New Castle, DE, USA).

### Cytotoxic Effects

The toxicity of freeze-dried tES was evaluated on the human breast cancer cell line, MDA-MB-231 (ATCC, Manassas, VA, USA). Briefly, 0.1×10⁶ cells were seeded in 12-well plates and cultured overnight. The following day, cells were treated with tES (30 mg/mL) or PBS as the control. The cells were incubated for 24 h at 37° C. and the cell viability was determined using the CellTiter-Glo® Luminescent Cell Viability Assay (Promega, Madison, WI, USA).

### Results

### Example 2: Expression and Purification of tES

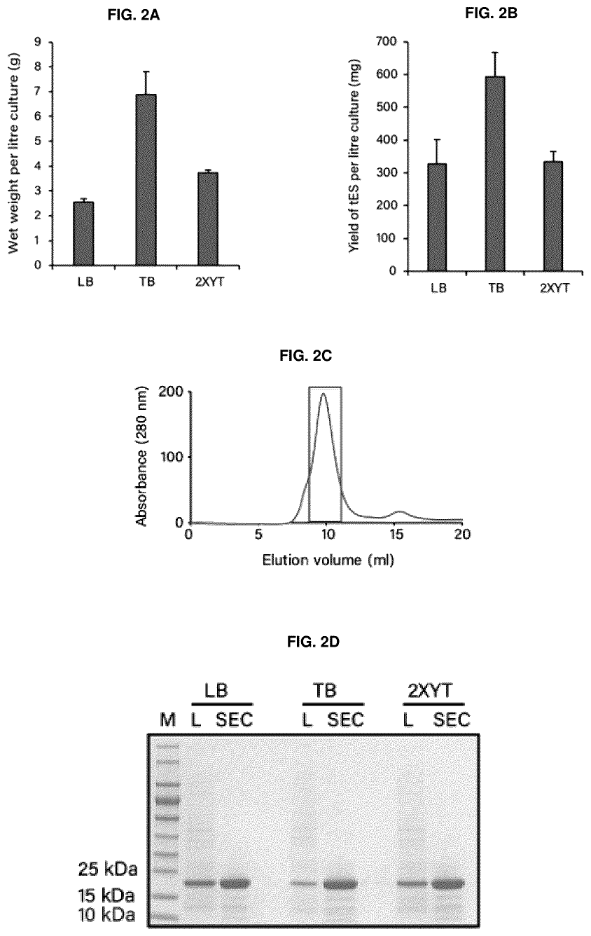

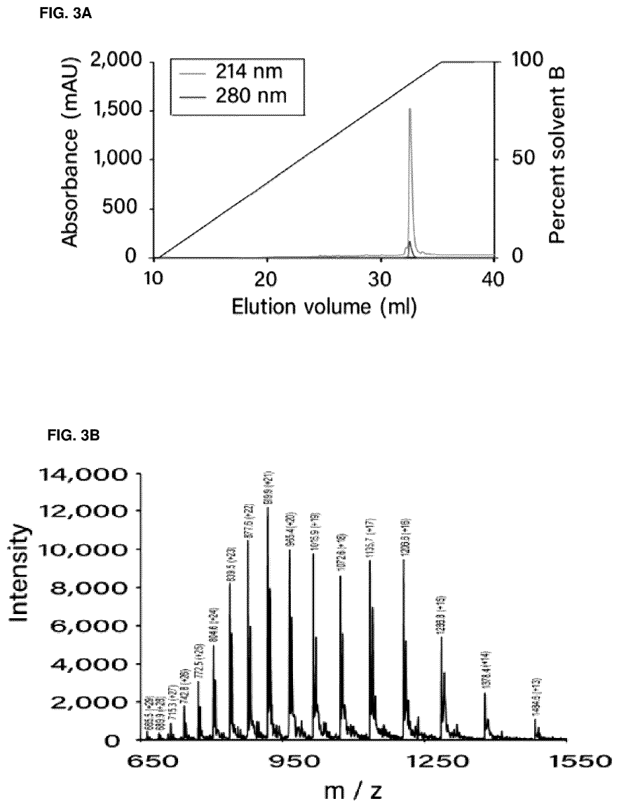

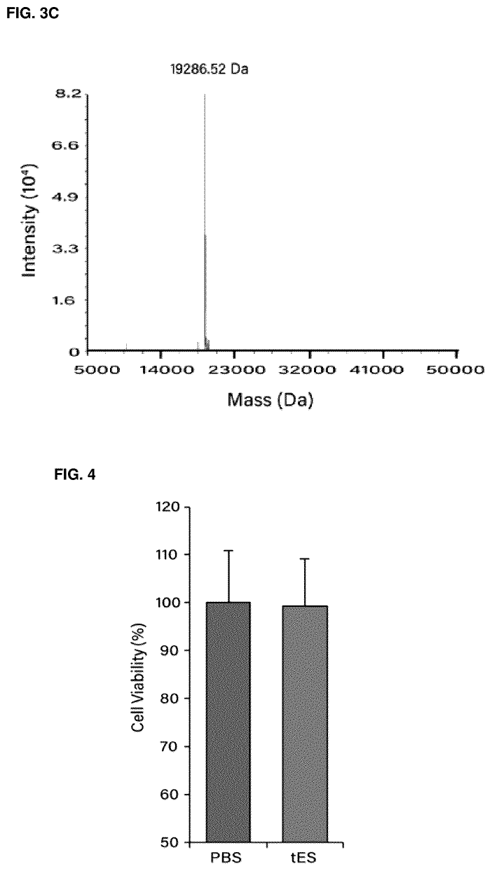

tES is characterized by a c-terminus truncation at Gln164 for extra internal space, internally facing mutations that create a net positive charge environment, and a Phe116His (F116H) mutation at the 3-fold symmetry axis for pH-dependent shell disassembly/assembly (Deshpande, S., et al., Nat. Commun. 8, 1-8 (2017)). Acknowledging the need for high production of biomass for future use of tES as a protein excipient, we systematically compared the cell density and protein yields per liter of bacterial culture using three different media, i.e., LB, TB, or 2XYT. Of the three, TB-grown cultures demonstrated a 2-3 fold increase in cell density and protein quantity for tES compared to LB or 2XYT grown cultures, the final purified yield estimated to be ˜600 mg per liter culture (FIGS. 2A, 2B). tES purification was achieved through a single-step chromatography involving SEC (FIG. 2C). tES eluted around 10 mL with a minor subunit fraction around 16 mL. The purity of tES from the highlighted peak was analyzed by SDS-PAGE with a single band corresponding to a subunit mass of ˜20 kDa (FIG. 2D). The SEC purified fraction was further analyzed in RP-HPLC followed by ESI-MS to determine the homogeneity and mass of tES. The RP-HPLC chromatography exhibited a single peak eluted at 90% solvent B (FIG. 3A). The protein showed multiple peaks of mass/charge (m/z) ratios ranging from +13 to +29 charges in ESI-MS. The observed mass of 19,286.52 Da was similar to the calculated mass of 19,287.13 Da from the amino acid sequence (FIGS. 3B, 3C). Additionally, the in vitro examination of purified tES was found to be non-toxic in a cell viability study (FIG. 4).

### Example 3: Lyophilization and Stability of Freeze-Dried Proteins

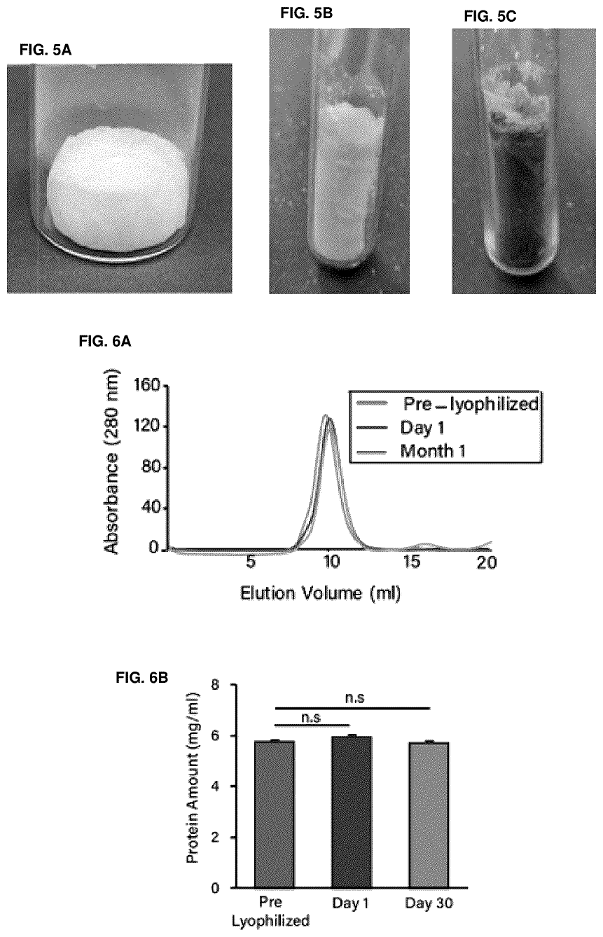

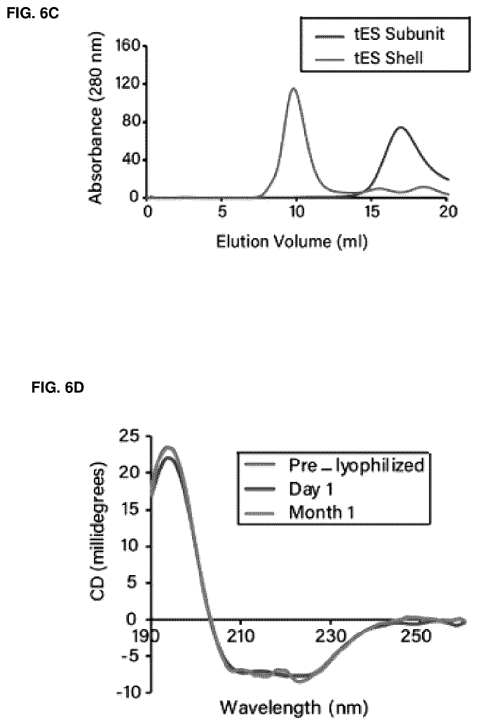

As an attempt to understand the stability of tES or its subunits in dry powder form, we subjected the proteins to lyophilization followed by storage at RT for up to 1 month. Prior to lyophilization, the proteins were buffer exchanged to ammonium bicarbonate or formate buffer as these are readily removed during the sublimation stage (Franks, F., Eur. J. Pharm. BioPharmaceutics 45, 221-229 (1998)). The proteins after lyophilization exhibited cake shrinkage without any signs of collapse or cracking (FIGS. 5A-5C). At both day 1 and 30, the reconstituted tES co-eluted at a predicted molecular mass of 480 kDa on a calibrated SEC column, consistent with that of pre-lyophilized samples, suggesting that the shell did not disintegrate after freeze-drying and storage (FIG. 6A). We also estimated the protein concentration after 1 month of storage and was found to be the same as the pre-lyophilized samples, indicating negligible protein loss (FIG. 6B). Similar to tES, the subunits after their storage at RT for 1 month assembled into shells with a predicted molecular mass at pH 8.0. Alternatively, they also maintained their monomeric stature based on the elution at pH 5.8 buffer (FIG. 6C). Additionally, we studied the CD spectra to observe any secondary conformational changes of the freeze-dried proteins during storage. The tES shells exhibited similar and comparable spectral patterns to the pre-lyophilized samples, with an intense minimum at 210 nm and 225 nm and maximum at 194 nm, typical of α-helical structure (FIG. 6D) (Micsonai, A., et al., Proc. Natl. Acad. Sci. USA 112, E3095-E3103 (2015)). Taken together, the results suggest the innocuous effects of freeze-drying on the structures of tES.

### Example 4: Nanoencapsulation Studies

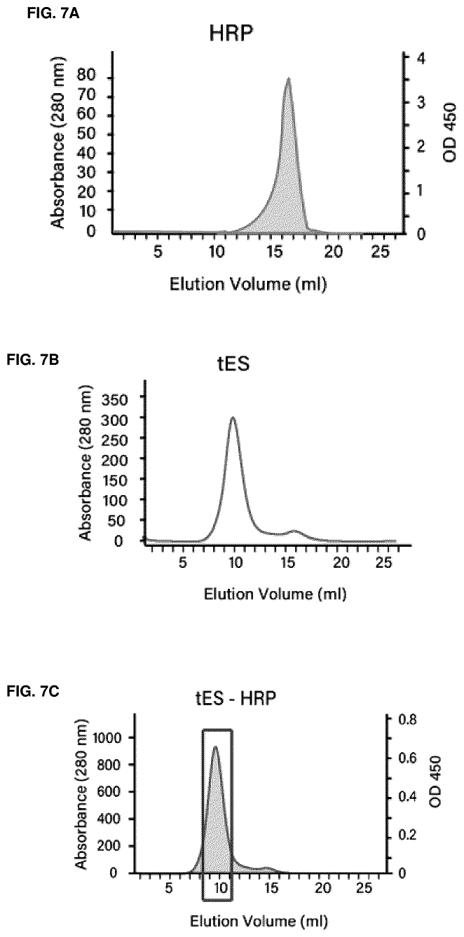

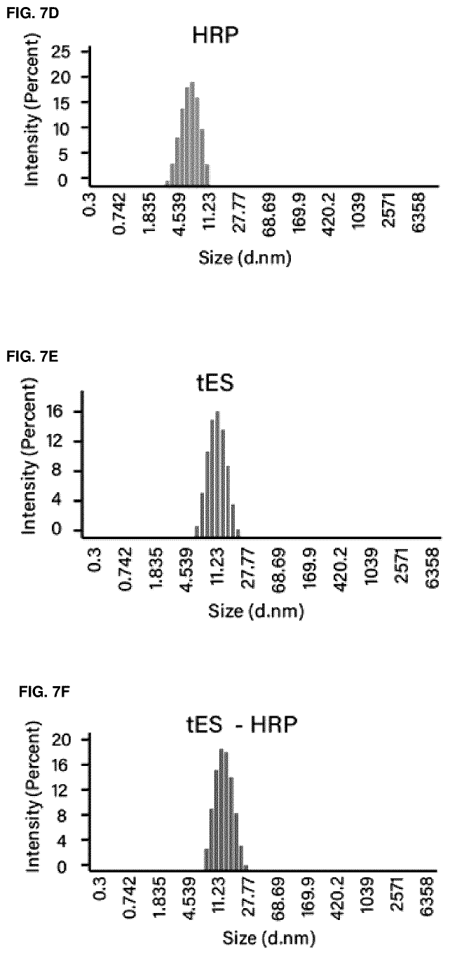

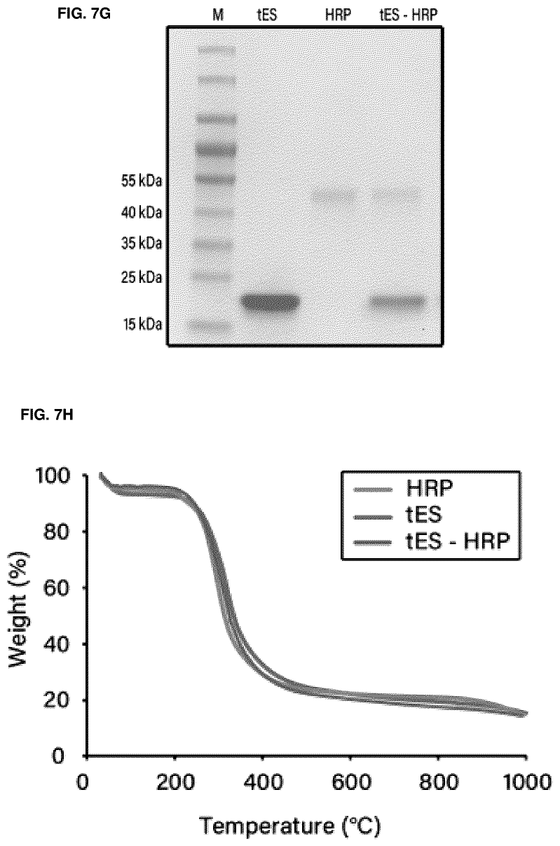

Previous studies from our group have shown that HRP covalently bound to a tES subunit suitably fits inside the positive cavity of tES (Deshpande, S., et al., Nat. Commun. 8, 1-8 (2017)). Based on this observation, we hypothesized that industrial HRP (Thermo Fisher Scientific, Waltham, MA, USA) could be diffusionally loaded into tES shells for lyophilization using a pH-mediated particle disassembly and assembly method. Encapsulation without any signs of aggregation was confirmed using both SEC and DLS. As expected, tES-HRP eluted as a peak at pH 8.0 with an anticipated diffusional diameter with HRP activity tracked at this peak (FIGS. 7A-7C). Moreover, DLS displayed similar hydrodynamic diameters of 15 nm for both tES and tES-HRP confirming the internalization of HRP. The free HRP had a calculated diameter of 6 nm. All the proteins analyzed had a polydispersity index <0.5 (FIGS. 7D-7F). Likewise, hydrodynamic diameters were consistent with the published literature (Fu, B., et al., Ann. Biomed. Eng. 25, 375-397 (1997); Sana, B., et al., J. Biol. Chem., 288, 32663-32672 (2013)). SDS gel electrophoresis of purified tES-HRP displayed two bands corresponding to tES subunits and HRP (FIG. 7G). We determined the encapsulation of two HRP molecules within the tES and further confirmed using analytical ultra-centrifugation (data not shown). Further, we determined the moisture content in each freeze-dried sample using TGA. The degradation temperatures were determined from the peak of derivative weight curves. For all the samples analyzed, there were two decomposition temperatures (Td) with the first one corresponding to the water loss and the second one linked to the degradation of the protein samples, as reported earlier (FIG. 7H) (Giovannelli, L., et al., Pharmaceuticals, 14, 262 (2021)). The average moisture content in all the freeze-dried samples is represented in Table 2.

TABLE 2
Decomposition temperatures (Td) and moisture
content (%) of freeze-dried samples.

| Sample | Td *(° C.) Td **(° C.) | Moisture content (%) |
|---|---|---|
| HRP | 55.5 ± 2.6 307.2 ± 0.1 | 4.6 ± 0.2 |
| tES | 59.7 ± 2.9 328.5 ± 3.0 | 5.5 ± 0.9 |
| tES-HRP | 59.4 ± 3.4 329.5 ± 1.9 | 4.4 ± 0.8 |

*First decomposition temperature.
**Second decomposition temperature.
All the analysis is from three independently processed samples and the values expressed as the mean ± S.D.

### Example 5: Nanoencapsulation Stabilizes HRP in Dry Form

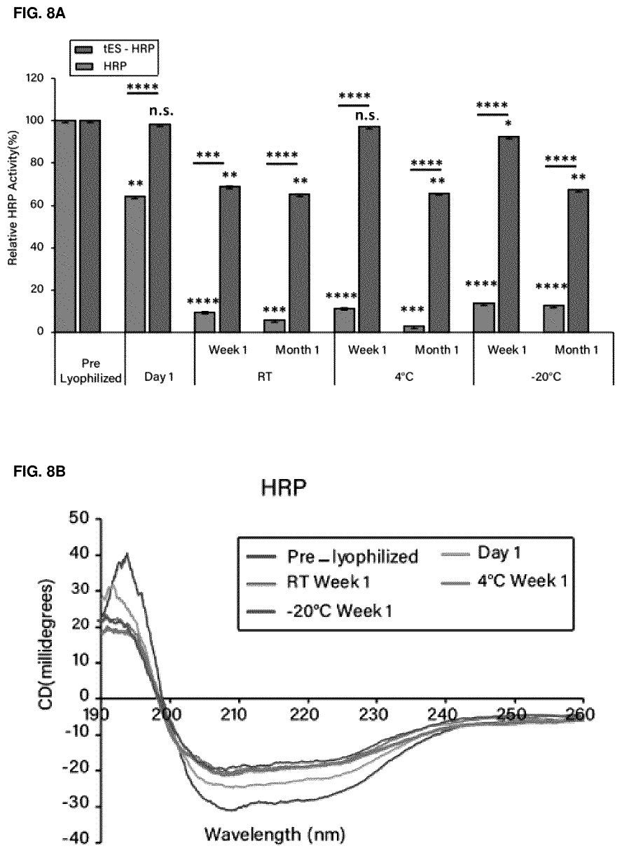

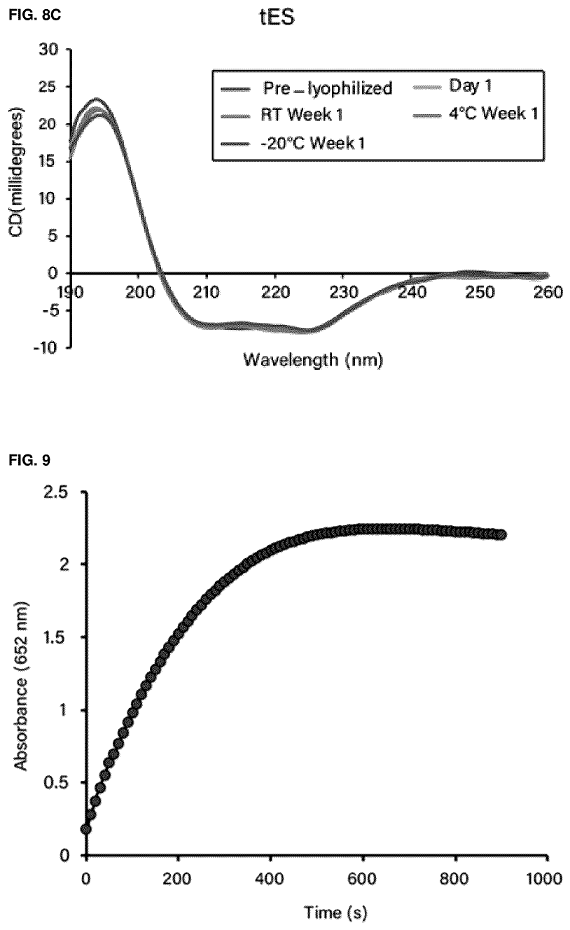

The freeze-dried tES-HRP displayed significant enzyme activity even after 1 month of storage. In all the conditions evaluated, the free HRP activity was reduced to ˜10% or less after 1 week/1 month storage period. Surprisingly, tES protected the HRP activity (˜70-99%) under all the storage conditions evaluated (FIG. 8A, Table 3).

TABLE 3
Average reads and standard deviations for HRP
activity from three independent experiments.

| | Reads Average | Reads Average | | Reads Average |
|---|---|---|---|---|
| | One Week | | | |
| | RT | 4° C. | | −20° C. |
| Lyophilized HRP | 0.54 ± 0.17 | 0.65 ± 0.08 | | 0.75 ± 0.08 |
| | [symbol] | | | |
| Lyophilized tES- | 3.92 ± 0.27 | 5.53 ± 0.18 | | 5.22 ± 0.13 |
| HRP | | 0.27 | | |
| | One Month | | | |
| | RT | 4° C. | | −20° C. |
| Lyophilized HRP | 0.33 ± 0.02 | 0.16 ± 0.02 | | 0.68 ± 0.02 |
| | | [symbol] | | |
| Lyophilized tES- | 3.71 ± 0.01 | 3.74 ± 0.01 | | 3.79 ± 0.01 |
| HRP | | 0.01 | | |
| | One Day | | | Pre-Lyophilized |
| Lyophilized HRP | 3.61 ± 0.04 | | HRP | 5.55 ± 0.33 |
| | [symbol] | | | [symbol] |
| Lyophilized tES- | 5.23 ± 0.11 | | tES | 5.72 ± 0.15 |
| HRP | 0.11 | | | 0.15 |

[symbol]

indicates data missing or illegible when filed

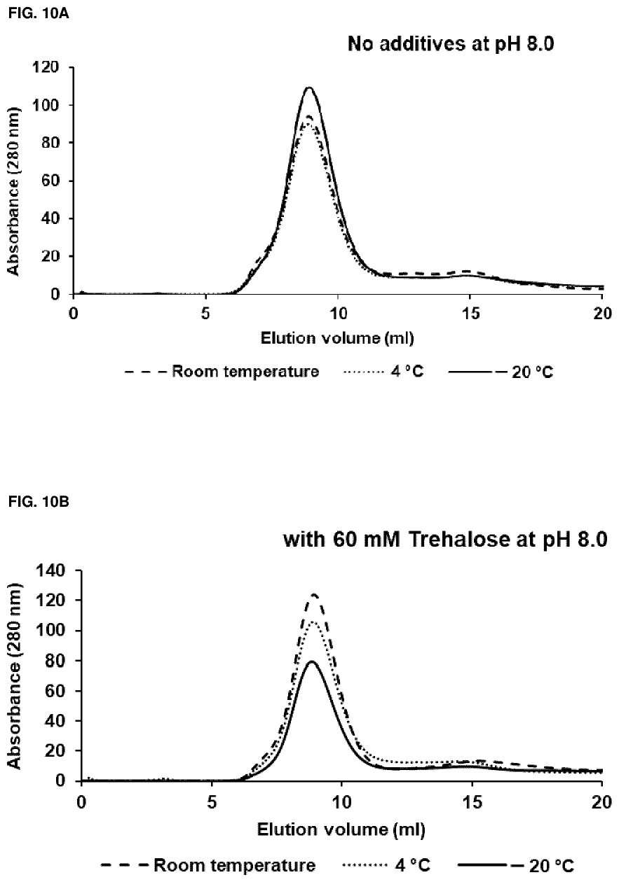

In addition, a time-course analysis of the freeze-dried tES-HRP activity was investigated (FIG. 9). The initial velocity (vₒ) expressed as a change in absorbance per unit of time (ΔA₆₅₂/min) was determined as 0.45/min. Two important observations were made from this study. Firstly, at RT storage, tES-based nanoencapsulation preserved the activity of commercially obtained HRP in spite of the required storage conditions of −20° C. Secondly, the encapsulated HRP activity was comparable to the pre-lyophilized sample, but free HRP activity was reduced to 70% immediately after freeze-drying. To explain this, we investigated the secondary structures of HRP and tES pre- and post-lyophilization. The CD spectrum of pre-lyophilized HRP was characterized by a double minimum at 209 nm and 221 nm and maxima at 194 nm, indicative of a predominant α-helical structure. After freeze-drying and subsequent storage, HRP exhibited changes in the CD spectra (FIG. 8B) which could be responsible for the significant reduction in the enzyme activity. A similar reduction in the enzyme activity associated with conformational changes is previously reported (Carrasquillo, K. G., et al., Biotechnol. Appl. Biochem. 31, 41-53 (2000); Roy, I., et al., Biotechnol. Appl. Biochem. 39, 165-177 (2004)). In tES, the freeze-drying had no observable effects on secondary structure as assessed by circular dichroism (FIG. 8C). Collectively, these observations suggest that tES acts as a strong lyoprotectant for the encapsulated enzyme.

### Example 6: Freeze-Dried tES are Stable Long-Term

For a successful nanoencapsulation, the tES needs to be disassembled/assembled using mild pH titrations. Also, for the long-term storage of the encapsulated molecule, the structural integrity and ability of tES to disassemble/assemble has to be evaluated.

### Methodology

The stability of the lyophilized tES was studied over a period of 8 months. The lyophilized protein was stored in three temperature conditions: Room Temperature (RT), 4° C., −20° C. with or without the commonly used excipients (60 mM trehalose or 60 mM sorbitol). The stability over time was evaluated through size-exclusion chromatography (SEC) and circular dichroism (CD) studies. SEC was used to study the ability of tES (2 mg/ml) to assemble or disassemble at alkaline (8.0) or acidic (5.6) pH, respectively. Samples were run on a Superdex 200 column equilibrated with either 50 mM ammonium bicarbonate buffer (pH 8.0) to test the assembly of the cage or a 50 mM ammonium acetate buffer (pH 5.6) to test the disassembly of the cage. To prepare the samples for CD spectroscopy, the lyophilized powder was reconstituted in 10 mM phosphate buffer (pH 8.0) at a concentration of 0.1 mg/ml and the measurement was taken across a range of 190 nm and 260 nm. The results of both the SEC and CD spectroscopy were plotted to assess the stability of the protein nanoparticle.

### Size-Exclusion Chromatography Studies

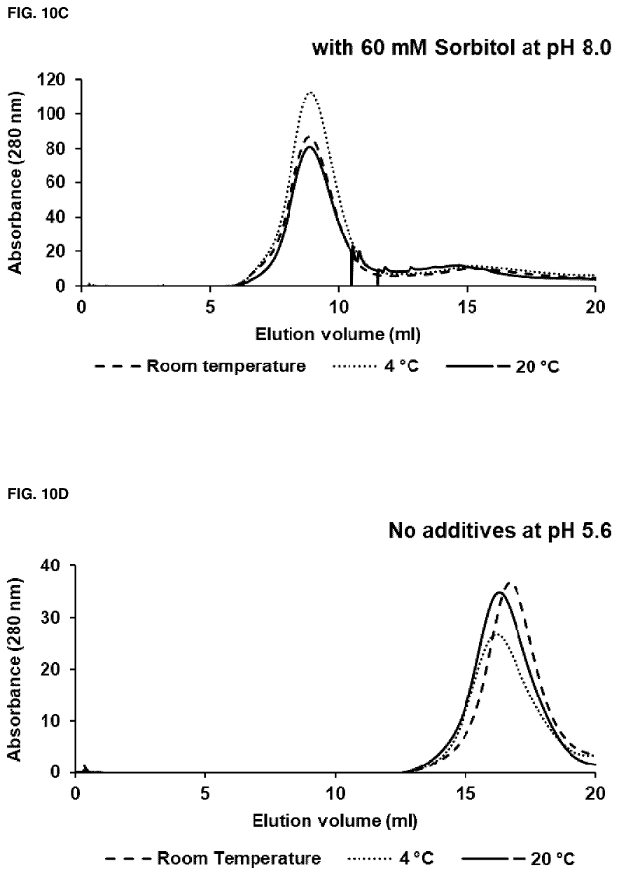

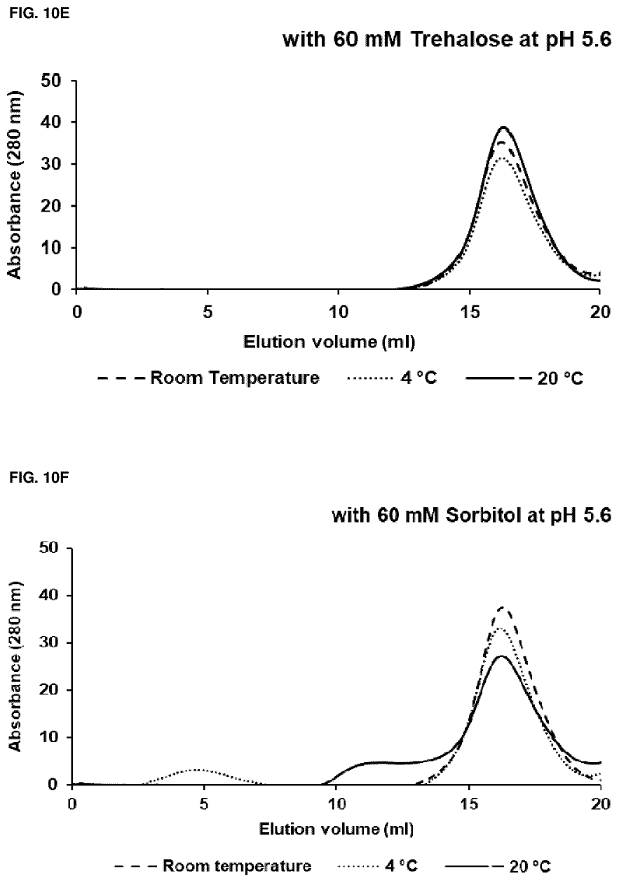

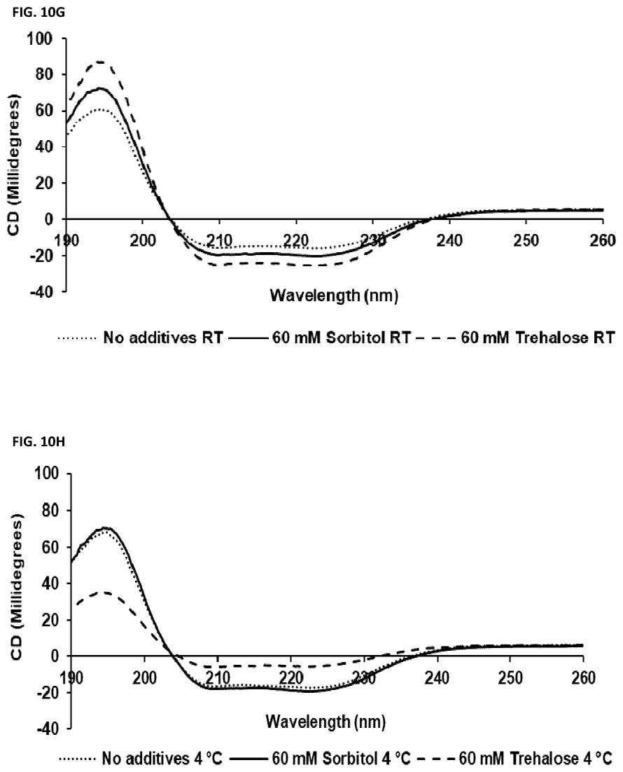

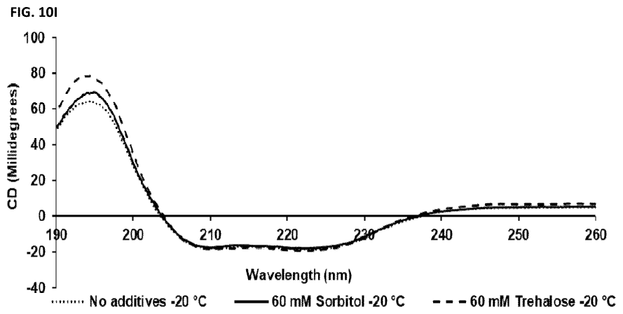

SEC showed that tES maintained its hydrodynamic volume and eluted as a fully assembled shell at pH 8.0 even after 8 months of storage (FIGS. 10A-10C) with or without added excipients. Further, tES efficiently disassembled under acidic conditions of pH 5.6 after 8 months of storage (FIGS. 10D-10F).

### CD Spectroscopy Studies

tES with or without the additives maintained its alpha helical structure after 8 months of storage at all the conditions evaluated (FIGS. 10G-10I), implying the stability of tES at RT even in the absence of commonly used excipients like trehalose or sorbitol. The alpha helix in CD spectroscopy is denoted by a maximum at ˜190 nm and two minima at ˜210 nm and ˜224 nm.

The results emphasize that tES as such is stable at room temperature and does not require commonly used excipients like trehalose or sorbitol during storage.

### SUMMARY

The objective of lyophilization is to produce a stable formulation in dry form for long-term storage and transportation. Overall, freeze-drying tES provided a stable, immobile environment within its charged internal cavity. Further, lyophilized tES retained protein activity during a one-month evaluation, having a near 100% recovery following aqueous reconstitution. Time-course experiments demonstrated that unprotected HRP lost 95% of activity after 1 month of lyophilized storage. In contrast, after encapsulation within tES nanoparticles, 70% of HRP activity was recovered, representing a surprising 14-fold improvement. This effect was reproducible across a range of storage temperatures.

HRP was selected due to its broad usage in both clinical and industrial applications such as therapeutics and diagnostics, biosensor systems, bioremediation, and biocatalysis. Additionally, the applicability of HRP in enzyme-prodrug therapy and the development of various diagnostic tools were acknowledged recently (Bonifert, G., et al., Cancer Med. 5, 1194-1203 (2016); Sheng, Y., et al., J. Nano-biotechnol. 17, 1-15 (2019)).

The results demonstrate a mild pH titration protocol for protein encapsulation, wherein the tES can be disassembled at pH 5.8 and assembled at pH 8.0, a range that is expected to be compatible with the majority of protein structures (Al-Bagmi, M. S., et al., Saudi J. Biol. Sci., 26, 301-307 (2019)). Likewise, recovery of the encapsulated proteins can be achieved through mild acidification of shells (Deshpande, S., et al., Nat. Commun. 8, 1-8 (2017)). HRP encapsulation was confirmed through SEC and DLS experiments (FIGS. 7A-7H). Furthermore, the encapsulated HRP has previously been shown to access its substrate through the 4.5 nm surface pores (FIG. 8A).

The nanoencapsulation process described herein is based on complementary charge pairing wherein the negatively charged proteins such as HRP are confined within a positive shell. The mechanism of tES protection of encapsulated protein, we hypothesize, is two-fold. In the freezing phase of lyophilization, ice crystals are presumed to have a denaturing effect via physical disruption of protein structure. A strong, thermostable cage that surrounds the protein may thus provide a physical barrier to this denaturation. Secondly, the internal surface of the shell is engineered to complement the surface charge of the encapsulated protein. Providing this energetic stabilization may further enhance storage stability. Similar to this, hydrogen bonds are known to stabilize the dry protein formulations with the use of excipients such as sugars (Chang, L. L., et al., J. Pharm. Sci., 94, 1427-1444 (2005); Mensink, M. A., et al., Eur. J. Pharm. Biopharm., 114, 288-295 (2017)).

A caveat of our study is that even though tES maintained HRP activity, we observed a ˜30% reduction in activity over the storage period. This could be attributed to the moisture-triggered structural changes and inactivation in the freeze-dried proteins. Similar de-stabilization due to moisture was observed during the storage of freeze-dried therapeutic proteins insulin and interleukin-2 (Roy, I., and Gupta, M. N., Biotechnol. Appl. Biochem., 39, 165-177 (2004); Duralliu, A., et al., Pharmaceutics, 12, 303 (2020)). We determined the residual moisture content in all the freeze-dried samples. TGA studies confirmed the presence of ˜4-5% of residual moisture in all the samples analyzed (Table 2). Hence, it is critical to maintain the moisture content within the desired ranges as an acceptable level may ensure stabilized protein activity, non-aggregation, and easy sample reconstitution when compared to very low or high residual moisture (Towns, J. K., J. Chromatogr. A, 705, 115-127 (1995); May, J. C., CRC Press: Boca Raton, FL; pp. 302-330 (2016)).

tES as a reagent is non-toxic to cell viability (FIG. 4), can be easily purified in high yield, stored lyophilized at RT for at least 8 months, and reused multiple times for disassembly/assembly using mild pH titrations. Thus, the dry formulations of tES encapsulated labile therapeutics can be advantageous in sustained release for systemic or local delivery when compared to polymeric or lipid-based systems. As tES based freeze-drying is independent of additives and homogenous on storage, they can function both as a cryoprotectant and lyoprotectant.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

### REFERENCES

Any listing or discussion of an apparently prior-published document in this specification should not necessarily be taken as an acknowledgement that such document is part of the state of the art or is common general knowledge.
Al-Bagmi, M. S.; Khan, M. S.; Ismael, M. A.; Al-Senaidy, A. M.; Bacha, A. B.; Husain, F. M.; Alamery, S. F. An efficient method-ology for the purification of date palm peroxidase: Stability comparison with horseradish peroxidase (HRP). Saudi J. Biol. Sci. 2019, 26, 301-307.
Bonifert, G.; Folkes, L.; Gmeiner, C.; Dachs, G.; Spadiut, O. Recombinant horseradish peroxidase variants for targeted cancer treatment. Cancer Med. 2016, 5, 1194-1203.
Carrasquillo, K. G.; Sanchez, C.; Griebenow, K. Relationship between conformational stability and lyophilization-induced structural changes in chymotrypsin. Biotechnol. Appl. Biochem. 2000, 31, 41-53.
Chang, L. L.; Shepherd, D.; Sun, J.; Ouellette, D.; Grant, K. L.; Tang, X. C.; Pikal, M. J. Mechanism of protein stabilization by sugars during freeze-drying and storage: Native structure preservation, specific interaction, and/or immobilization in a glassy matrix? J. Pharm. Sci. 2005, 94, 1427-1444.
Deshpande, S.; Masurkar, N. D.; Girish, V. M.; Desai, M.; Chakraborty, G.; Chan, J. M.; Drum, C. L. Thermostable exoshells fold and stabilize recombinant proteins. Nat. Commun. 2017, 8, 1-8.
Duralliu, A.; Matejtschuk, P.; Stickings, P.; Hassall, L.; Tierney, R.; Williams, D. R. The influence of moisture content and temperature on the long-term storage stability of freeze-dried high concentration immunoglobulin G (IgG). Pharmaceutics 2020, 12, 303.
Faghihi, H.; Khalili, F.; Amini, M.; Vatanara, A. The effect of freeze-dried antibody concentrations on its stability in the presence of trehalose and hydroxypropyl-β-cyclodextrin: A box-behnken statistical design. Pharm. Dev. Technol. 2017, 22, 724-732.
Franks, F. Freeze-drying of bioproducts: Putting principles into practice. Eur. J. Pharm. BioPharmaceutics 1998, 45, 221-229.
Franzé, S.; Selmin, F.; Samaritani, E.; Minghetti, P.; Cilurzo, F. Lyophilization of liposomal formulations: Still necessary, still challenging. Pharmaceutics 2018, 10, 139.
Fu, B.; Curry, F.-R. E.; Adamson, R. H.; Weinbaum, S. A model for interpreting the tracer labeling of interendothelial clefts. Ann. Biomed. Eng. 1997, 25, 375-397.
Giovannelli, L.; Milanesi, A.; Ugazio, E.; Fracchia, L.; Segale, L. Effect of methyl-β-cyclodextrin and trehalose on the freeze-drying and spray-drying of sericin for cosmetic purposes. Pharmaceuticals 2021, 14, 262.
Guex, N.; Peitsch, M. C.; Schwede, T. Automated comparative protein structure modeling with SWISS-MODEL and Swiss-PdbViewer: A historical perspective. Electrophoresis 2009, 30, S162-S173.
Hudson, K. L.; Bartlett, G. J.; Diehl, R. C.; Agirre, J.; Gallagher, T.; Kiessling, L. L.; Woolfson, D. N. Carbohydrate-aromatic interactions in proteins. J. Am. Chem. Soc. 2015, 137, 15152-15160.
Johnson, E.; Cascio, D.; Sawaya M. R.; Gingery, M.; Schroeder, I.; Crystal structures of a tetrahedral open pore ferritin from the hyperthermophilic archaeon Archaeoglobus fulgidus. Structure. 2005, 13, 637-648.
Kadoya, S.; Fujii, K.; Izutsu, K.-I.; Yonemochi, E.; Terada, K.; Yomota, C.; Kawanishi, T. Freeze-drying of proteins with glass-forming oligosaccharide-derived sugar alcohols. Int. J. Pharm. 2010, 389, 107-113.
Kamerzell, T. J.; Esfandiary, R.; Joshi, S. B.; Middaugh, C. R.; Volkin, D. B. Protein-excipient interactions: Mechanisms and biophysical characterization applied to protein formulation development. Adv. Drug Deliv. Rev. 2011, 63, 1118-1159.
Li, S.; Patapoff, T. W.; Overcashier, D.; Hsu, C.; Nguyen, T. H.; Borchardt, R. T. Effects of reducing sugars on the chemical stability of human relaxin in the lyophilized state. J. Pharm. Sci. 1996, 85, 873-877.
May, J. C. Regulatory control of freeze-dried products: Importance and evaluation of residual moisture. In Freeze-Drying/Lyophilization of Pharmaceutical and Biological Products; CRC Press: Boca Raton, FL, USA, 2016; pp. 302-330.
Mensink, M. A.; Frijlink, H. W.; van der Voort Maarschalk, K.; Hinrichs, W. L. How sugars protect proteins in the solid state and during drying (review): Mechanisms of stabilization in relation to stress conditions. Eur. J. Pharm. Biopharm. 2017, 114, 288-295.
Micsonai, A.; Wien, F.; Kernya, L.; Lee, Y.-H.; Goto, Y.; Refrégiers, M.; Kardos, J. Accurate secondary structure prediction and fold recognition for circular dichroism spectroscopy. Proc. Natl. Acad. Sci. USA 2015, 112, E3095-E3103.
Picco, A. S.; Mondo, G. B.; Ferreira, L. F.; de Souza, E. E.; Peroni, L. A.; Cardoso, M. B. Protein corona meets freeze-drying: Overcoming the challenges of colloidal stability, toxicity, and opsonin adsorption. Nanoscale 2021, 13, 753-762.
Roy, I.; Gupta, M. N. Freeze-drying of proteins: Some emerging concerns. Biotechnol. Appl. Biochem. 2004, 39, 165-177.
Sadeghi, S.; Deshpande, S.; Vallerinteavide Mavelli, G.; Aksoyoglu, A.; Bafna, J.; Winterhalter, M.; Kini, R. M.; Lane, D. P.; Drum, C. L. A general approach to protein folding using thermostable exoshells. Nat. Commun. 2021, 12, 1-15.
Sana, B.; Johnson, E.; Le Magueres, P.; Criswell, A.; Cascio, D.; Lim, S. The role of nonconserved residues of Archaeoglobus fulgidus ferritin on its unique structure and biophysical properties. J. Biol. Chem. 2013, 288, 32663-32672.
Sheng, Y.; Wang, K.; Lu, Q.; Ji, P.; Liu, B.; Zhu, J.; Liu, Q.; Sun, Y.; Zhang, J.; Zhou, E.-M. Nanobody-horseradish peroxidase fusion protein as an ultrasensitive probe to detect antibodies against Newcastle disease virus in the immunoassay. J. Nano-biotechnol. 2019, 17, 1-15.
Towns, J. K. Moisture content in proteins: Its effects and measurement. J. Chromatogr. A 1995, 705, 115-127.
Voss, N. R.; Gerstein, M. 3V: Cavity, channel and cleft volume calculator and extractor. Nucleic Acids Res. 2010, 38, W555-W562.
Xu, H.; Templeton, A. C.; A Reed, R. Quantification of 5-HMF and dextrose in commercial aqueous dextrose solutions. J. Pharm. Biomed. Anal. 2003, 32, 451-459.

1. A method of reducing structural and/or functional loss of a labile molecule during storage, the method comprising;
a) encapsulating a labile molecule within an engineered thermostable ferritin assembly which comprises at least one modified subunit, wherein the at least one modified ferritin subunit comprises the amino acid sequence set forth in SEQ ID NO: 1, except that the modified ferritin subunit lacks residues 165-173 of SEQ ID NO: 1, comprises a F116H substitution for pH-dependent shell disassembly/assembly, and zero or more amino acid substitutions at one or more positions selected from the group comprising E65, E128, E131, and D138 of SEQ ID NO: 1; and
b) freeze-drying the assembly.

2. The method of claim 1, wherein the labile molecule is a macromolecule.

3. The method of claim 1, wherein pH-dependent shell disassembly/assembly is effected in the range between pH 5.6 and pH 8.

4. The method of claim 1, wherein the thermostable ferritin assembly has internal charge complementarity with the labile molecule.

5. The method of claim 1, wherein the thermostable ferritin assembly is characterized by:
i) a net internal positive (+) charge due to an amino acid substitution at any two or more positions of said ferritin assembly subunit selected from E65K, E128K, E131K, and D138A; or
ii) a net internal neutral (+/−) charge due to amino acid substitutions E65Q and D138A of said ferritin assembly subunit; or
iii) a net internal negative (−) charge due to zero amino acid substitutions at said ferritin assembly subunit positions E65, E128, E131 and D138.

6. The method of claim 5, wherein said thermostable ferritin assembly modified subunit comprises the amino acid sequence set forth in SEQ ID NO: 5, SEQ ID NO: 6, or SEQ ID NO: 7, or a variant thereof which retains the ability to assemble.

7. The method of claim 1, wherein prior to step b) the assembly is chromatographed on a column equilibrated with a volatile salt buffer at about pH 8.0.

8. The method of claim 7, wherein the buffer is ammonium bicarbonate or formate buffer.

9. The method of claim 7, wherein the buffer is about 10 mM ammonium bicarbonate.

10. The method of claim 1, wherein the freeze-drying is performed for a period of about 48 h or more, depending on the volume of the assembly.

11. The method of claim 1, wherein the labile macromolecule is a polypeptide or polynucleic acid.

12. The method of claim 1, wherein said thermostable ferritin assembly acts as an excipient for freeze-drying.

13. A kit comprising thermostable ferritin assembly modified subunits, as defined in claim 1, to be used in the method of claim 1.

14. A freeze-dried composition comprising a labile molecule encapsulated within an engineered thermostable ferritin assembly as defined in claim 1.

15. The freeze-dried composition of claim 14, produced by the method of claim 1.

16. The freeze-dried composition of claim 14, for use in the prophylaxis or treatment of disease in a subject.

17. The freeze-dried composition of claim 14, wherein the encapsulated labile molecule comprises an enzyme for conversion of a prodrug.

18. The freeze-dried composition of claim 14, further comprising one or more additional therapeutic agents.

19. (canceled)

20. A method of treatment or prophylaxis comprising administering to a subject in need of such treatment or prophylaxis an efficacious amount of a freeze-dried composition of claim 14.
